(19) United States
(12) Patent Application Publication
(10) Pub. No.: US 2024/0024234 A1
(43) Pub. Date: Jan. 25, 2024

(54) Formulations for Delivery of Bioactive Agents

(71) Applicant: Royal Melbourne Institute of Technology, Melbourne, Victora (AU)

(72) Inventors: Nazim Nassar, Bundoora, Victoria (AU); Taghrid Istivan, Bundoora, Victoria (AU); Stefan Kasapis, Melbourne, Victoria (AU)

(73) Assignee: Royal Melbourne Institute of Technology, Melbourne, Victora (AU)

(21) Appl. No.: 18/266,064

(22) PCT Filed: Dec. 10, 2021
(86) PCT No.: PCT/AU2021/051477

§ 371 Date: Jun. 8, 2023

(30) Foreign Application Priority Data
Dec. 11, 2020 (AU) 2020904616

(51) Int. Cl. A61K 9/06; A61K 47/12; A61K 47/18; A61K 47/38; A61K 38/40

(52) U.S. Cl. CPC A61K 9/06; A61K 47/12; A61K 47/18; A61K 47/38; A61K 38/40

(57) ABSTRACT

The present invention relates to formulations for the delivery of bioactive agents, and methods of preparing the formulations. More specifically, the present invention relates to a formulation for delivery of a bioactive agent comprising a hydrogel matrix that is the product of a reaction between hydroxypropyl methylcellulose (HPMC), acrylic acid (AAc) and N,N′-methylenebisacrylamide (MBA), and methods of preparing the formulations.

This application claims priority from Australian Provisional Patent Application 2020904616 filed on 11 Dec. 2020, the contents of which are to be taken as incorporated herein by this reference.

### TECHNICAL FIELD

The present invention relates to formulations for the delivery of bioactive agents, and methods of preparing the formulations.

### BACKGROUND OF INVENTION

Conventional drug administration often requires high dosages or repeated administration to maintain therapeutic levels in a patient. This can compromise patient compliance, efficacy and, and can result in severe side effects and even toxicity owing to high doses.

Oral administration, which is the most common approach for delivering pharmaceuticals, is frequently limited by poor bioavailability, poor targeting and short circulation times. For example, acid sensitive compounds such as protein and peptide drugs will hydrolyse in the high acidic stomach environment before reaching the small intestine where they can be absorbed into the bloodstream, leading to poor bioavailability and efficacy.

The parenteral route is currently the most common method of administration of these biotherapeutics. This usually requires frequent injections and consequently reduces patient compliance. Nasal sprays have also been used but come with the drawbacks of low bioavailability and irritation to nasal mucosa, which have forced some of these treatments off the market. Some hormonal replacement therapy is delivered topically through patches, but these frequently cause skin irritation and hard to keep it sticky on the skin for a week.

Effective oral delivery of such compounds is desirable in order to increase patient compliance, reduce cost and reduce the number of doses. Research in drug delivery has therefore focused efforts on achieving controlled and local drug delivery using nanostructured systems such as liposomes, nanoparticles, membranes and hydrogels. Hydrogels are highly absorbent networks of crosslinked polymer chains, sometimes found as a colloidal gel in which water is the dispersion medium.

Hydroxypropyl methylcellulose (HPMC) is a polysaccharide with amphiphilic properties that has been used in the pharmaceutical industry for over 50 years as a binder, filler, suspending and emulsifying agent. HPMC's hydrophilic moiety provides high swellability leading to a significant loading capacity with a prolonged time of release. Meanwhile, the hydrophobicity of HPMC allows the monomer to self-assemble, which yields a suspension of nanoparticles with a considerable encapsulation property. These nanoparticles can reach biological environments (such as the bloodstream or cells) and offer protected and prolonged release of their cargo, following the presence of a desired stimulus such as pH, temperature or biochemical catalysts at the expected releasing site. HPMC is also compatible with a wide range of different drugs and it is chemically stable with global regulatory acceptance. However, HPMC dissolves at a rapid rate, and therefore makes it difficult to follow the diffusion-controlled drug release mechanism in real product situations.

A previously described system used hybrid nanogels of HPMC and polyacrylic acid (PAA) to encapsulate insulin and appeared to show the ability of the ingested new formula to maintain healthy levels of blood sugar readings in diabetic mice. Nanogels are nanoparticles that are defined by their predominant spherical morphology and physical softness. However, this system faces challenges regarding the release profile of the active ingredient from the biopolymer, as well as the stability of the nanogels, which means this product has not yet reached the market.

Furthermore, other previously described HPMC-based hydrogel systems have several problems such as rapid dissolution rate which makes it difficult to follow the diffusion-controlled drug release mechanism in real product situations. These previously described systems also have shown poor mechanical strength and properties that cannot be tuned or predicted as needed.

There is therefore an ongoing need for improved HPMC-based materials which address some or all of these drawbacks and can be used to safely and effectively deliver bioactive agents to their targets.

A reference herein to a patent document or other matter which is given as prior art is not to be taken as an admission that the document or matter was known or that the information it contains was part of the common general knowledge as at the priority date of any of the claims.

### SUMMARY OF INVENTION

The inventors have surprisingly developed a HPMC-based hydrogel that can be used in formulations for the delivery of a wide range of bioactive agents in various settings.

In a first aspect the present invention provides a hydrogel that is the product of a reaction between hydroxypropyl methyl cellulose (HPMC) and acrylic acid (AAc) crosslinked with N,N′-methylenebisacrylamide (MBA).

In a second aspect present the invention provides hydrogel delivery system for delivery of a bioactive agent comprising a hydrogel matrix that is the product of a reaction between hydroxypropyl methylcellulose (HPMC), acrylic acid (AAc) and N,N′-methylenebisacrylamide (MBA).

One application of this delivery system is an oral delivery system for bioactive agents for the treatment of infectious disease, metabolic disorders, and some cancers. It also has application in wound care, for both humans and animals, and in the delivery of agrochemical compounds to plants and trees.

In a third aspect the present invention provides a method for synthesizing a hydroxypropyl methyl cellulose-acrylic acid (HPMC-AAc) hydrogel matrix, said method comprising the steps of: preparing a solution of HPMC in aqueous solvent; adding AAc to the solution of HPMC to form a reaction mixture; adding N,N′-methylenebisacrylamide (MBA) to the reaction mixture; adding a radical initiator to the reaction mixture to initiate polymerisation; allowing polymerisation to proceed for 4 hours at 38° C. to form a hydrogel matrix solution; incubating the hydrogel matrix solution in a drying atmosphere at 38° C. to isolate the hydrogel matrix.

This method allows for the flexible and scalable synthesis of a hydrogel matrix with tuneable properties that has application in the delivery of bioactive agents to a variety of targets.

The hydrogels of the present invention provide controlled release of the bioactive agents. There is no dumping factor and the hydrogels provide maintenance of optimal bioavailability over a long period.

The hydrogels of the present invention are pH responsive. There is little or no relaxation of the hydrogel matrix in an acidic environment, providing high protection for the bioactive payload in an environment such as the stomach.

The hydrogels of the present invention have great swellability which release over long periods. This feature is useful for the systemic delivery of bioactive compounds and for wound treatment.

The hydrogels of the present invention are biocompatible, having no interactions with the cargo or the target site to be treated. The hydrogels of the present inventions are biodegradable, breaking down to be safely excreted from the body. They also show little or no immunogenicity, compared with previously described biopolymers such as gelatin or biopolymers from animal sources.

The hydrogels of the present invention are also multipurpose and find applicability in a wide range of treatment scenarios. These include the oral administration of drugs that cannot otherwise be readily administered orally, application in wound care for both humans and animals, and in the delivery of agrochemical compounds to plants and trees. As well as improved bioavailability, the hydrogels of the present invention have the potential to improve patient compliance by providing oral delivery of drugs that would other have to be administered parenterally.

The hydrogels of the present invention demonstrate distinctive glass transitional behaviour with changing temperature altering their mechanical and thermodynamic properties. This state transition is pinpointed by the concept of the glass transition temperature (T_{g}) and allows the hydrogels of the present invention to enhance stability and to prolong shelf life of bioactive agents during storage.

The hydrogels of the present invention are also low-cost, being made from abundant, non-toxic ingredients in a manner that is easy to manufacture at scale. The ingredients are also widely accepted by religious and social groups (such as communities who follow kosher and halal regimes) which increases the potential number of users by around 1.5 billion people.

Further aspects of the invention appear below in the detailed description of the invention.

### BRIEF DESCRIPTION OF DRAWINGS

Embodiments of the invention will herein be illustrated by way of example only with reference to the accompanying drawings in which:

FIG. 1 is an FTIR spectra of AAc (acrylic acid), HPMC (hydroxypropyl methyl cellulose), and the prepared HPMC:AAc hydrogels (1:7, 1:6, 1:5, 1:4, 1:3, arranged successively downwards).

FIG. 2 is an X-ray diffractogram of the prepared HPMC:AAc hydrogels (1:7, 1:6, 1:5, 1:4, 1:3, arranged successively upwards).

FIG. 3 is a scanning electron micrograph (SEM) image of the HPMC:AAc hydrogel (1:3).

FIG. 4 shows the thermal profiles of G′, G″ and tan δ for the HPMC-AAc hydrogel (1:5).

FIG. 5 shows the frequency variation of: (a) G′. The bottom curve is taken at 48° C. (□); other curves successively upward, 44° C. (⋄), 40° C. (Δ), 36° C. (□), 32° C. (□), 28° C. (−), 24° C. (—), 20° C. (∘), 16° C. (+), 12° C. (▪), 8° C. (♦), 4° C. (▴), 0° C. (•), respectively.

FIG. 6 shows the frequency variation of G″. Bottom curve is taken at 48° C. (□); other curves successively upward, 44° C. (⋄), 40° C. (Δ), 36° C. (□), 32° C. (□), 28° C. (−), 24° C. (—), 20° C. (∘), 16° C. (+), 12° C. (▪), 8° C. (♦), 4° C. (♦), 0° C. (•), respectively.

FIG. 7 shows the real G′p (•) and imaginary G″p (∘) parts of the complex shear modulus for HPMC-AAc reduced to 20° C. and plotted logarithmically against reduced frequency (ωaT) utilising the mechanical spectra of FIG. 5.

FIG. 8 shows the logarithm of the reduction factor, α_{T}, for HPMC-AAc hydrogel plotted against temperature from the data of the master curve in FIG. 7.

FIG. 9 is a differential scanning calorimetric (DSC) thermogram of the prepared HPMC-AAc hydrogels (1:7, 1:6, 1:5, 1:4, 1:3, arranged successively upwards).

FIG. 10 is a thermo gravimetric analysis (TGA) of the prepared HPMC-AAc hydrogels (1:7, 1:6, 1:5, 1:4, 1:3, arranged successively upwards), and HPMC powder.

FIG. 11 is an image showing dry HPMC-AAc (1:7).

FIG. 12 is an image showing HPMC-AAc (1:7) after 24 h incubation in PBS at pH7.4.

FIG. 13 is an image showing HPMC-AAc (1:7) after 24 h incubation in PBS at pH6.

FIG. 14 is an image showing HPMC-AAc (1:7) after 24 h incubation in PBS at pH2.5.

FIG. 15 is a comparison chart of swelling ratios of different HPMC-AAc matrices as a function of time at pH 7.4.

FIG. 16 is a comparison chart of swelling ratios of different HPMC-AAc matrices as a function of time at pH 6.

FIG. 17 is a comparison chart of swelling ratios of different HPMC-AAc matrices as a function of time at pH 2.5.

FIG. 18 is a comparison chart showing fractional swelling of the hydrogel matrices over extended period at pH 7.4.

FIG. 19 is a plot using initial fractional swelling (8 hours) to determine the type of swelling by employing the Power Low Equation Mt/M∞=Ktⁿ.

FIG. 20 is a series of infinity microscopic images of (a) HPMC-AAc 1:7, (B) HPMC-AAc 1:5, AND (C) HPMC-AAc 1:3 at equilibrium of swelling (96 h) (10× objective lens).

FIG. 21 is a comparison chart showing the effect of the HPMC-AAc 1:7 mass ratio on the peptide diffusion (absorption levels) at different pH levels.

FIG. 22 is a comparison chart showing the effect of the HPMC-AAc 1:5 mass ratio on the peptide diffusion (absorption levels) at different pH levels.

FIG. 23 is a comparison chart showing the effect of the HPMC-AAc 1:3 mass ratio on the peptide diffusion (absorption levels) at different pH levels.

FIG. 24 is a comparison chart showing the peptide release by different HPMC-AAc matrices in the vicinity of pH=7.4.

FIG. 25 is a comparison chart showing the cumulative fractional diffusion of peptide-x analogue over prolonged period.

FIG. 26 is a plot using the initial fractional diffusion (first 8 hours) to determine the type of diffusion by employing the Power Low equation Mt/M∞=Ktⁿ.

FIG. 27 is a comparison chart showing cell viability of human keratinocyte cells (HaCat) following prolonged incubation with HPMC-AAc (1:7) using PrestoBlue methodology.

FIG. 28 is a comparison chart showing cell viability of human epithelial colorectal adenocarcinoma cells (CaCo2) following Prolonged incubation with HPMC-AAc (1:7) using PrestoBlue methodology.

FIG. 29 is a comparison chart showing optical density (O.D.600) readings for Staphylococcus aurous as a response to the released peptide-x from HPMC-AAc biopolymer over an elongated period.

FIG. 30 is a comparison chart showing optical density (O.D.600) readings for Pseudomonas aeruginosa as a response to the released peptide-x from HPMC-AAc biopolymer over an elongated period.

### DETAILED DESCRIPTION

Before describing the present invention in detail, it is to be understood that the terminology used herein is for the purpose of describing embodiments only and is not intended to be limiting.

Unless specifically defined otherwise, all technical and scientific terms used herein shall be taken to have the same meaning as commonly understood by one of ordinary skill in the art.

As used in this specification and the claims, the singular forms “a”, “an” and “the” include plural referents unless the content clearly dictates otherwise. Thus, for example, reference to a “bioactive agent” includes a combination of two or more such bioactive agents.

Throughout the description and claims of the specification the word “comprise” and variations of the word, such as “comprising” and “comprises”, is not intended to exclude other additives, components, integers or steps. As used herein, “comprises” means “includes”. Thus, “comprising A or B,” means “including A, B, or A and B,” without excluding additional elements.

As used herein, the term “bioactive agent” as used herein refers to a drug, protein, hormone, peptide or other compound that has a biological activity, related to its ability to modulate one or more metabolic processes of humans, animals or plants.

An HPMC:AAc Hydrogel

In one embodiment, the present invention relates to a hydrogel that is the product of a reaction between hydroxypropyl methyl cellulose (HPMC) and acrylic acid (AAc) crosslinked with N,N′-methylenebisacrylamide (MBA).

In preferred embodiments of the hydrogel of the invention, the mass ratio of hydroxypropyl methylcellulose to acrylic acid (HPMC:AAc) is between 1:3 and 1:10. More preferably, the mass ratio of hydroxypropyl methylcellulose to acrylic acid (HPMC:AAc) is from 1:3 to 1:7. In one preferred embodiment, the mass ratio of hydroxypropyl methylcellulose to acrylic acid (HPMC:AAc) is 1:3. In another preferred embodiment, the mass ratio of hydroxypropyl methylcellulose to acrylic acid (HPMC:AAc) is 1:4. In a further preferred embodiment, the mass ratio of hydroxypropyl methylcellulose to acrylic acid (HPMC:AAc) is 1:5. In yet another preferred embodiment, the mass ratio of hydroxypropyl methylcellulose to acrylic acid (HPMC:AAc) is 1:6. In another preferred embodiment, the mass ratio of hydroxypropyl methylcellulose to acrylic acid (HPMC:AAc) is 1:7.

In further preferred embodiments, the mass ratio of crosslinker to acrylic acid (MBA:AAc) is between 0.01 and 0.10, and more preferably around 0.05. In one preferred embodiment, the mass ratio of crosslinker to acrylic acid (MBA:AAc) is 0.01. In a further preferred embodiment, the mass ratio of crosslinker to acrylic acid (MBA:AAc) is 0.02. In another preferred embodiment, the mass ratio of crosslinker to acrylic acid (MBA:AAc) is 0.03. In another preferred embodiment, the mass ratio of crosslinker to acrylic acid (MBA:AAc) is 0.04. In a more preferred embodiment, the mass ratio of crosslinker to acrylic acid (MBA:AAc) is 0.05. In another preferred embodiment, the mass ratio of crosslinker to acrylic acid (MBA:AAc) is 0.06. In a further preferred embodiment, the mass ratio of crosslinker to acrylic acid (MBA:AAc) is 0.07. In another preferred embodiment, the mass ratio of crosslinker to acrylic acid (MBA:AAc) is 0.08. In a further preferred embodiment, the mass ratio of crosslinker to acrylic acid (MBA:AAc) is 0.09. In another preferred embodiment, the mass ratio of crosslinker to acrylic acid (MBA:AAc) is 0.10.

In a preferred embodiment, the mass ratio of hydroxypropyl methylcellulose to acrylic acid (HPMC:AAc) is 1:3 and the mass ratio of crosslinker to acrylic acid (MBA:AAc) is around 0.05. In another preferred embodiment, the mass ratio of hydroxypropyl methylcellulose to acrylic acid (HPMC:AAc) is 1:4 and the mass ratio of crosslinker to acrylic acid (MBA:AAc) is around 0.05. In another preferred embodiment, the mass ratio of hydroxypropyl methylcellulose to acrylic acid (HPMC:AAc) is 1:5 and the mass ratio of crosslinker to acrylic acid (MBA:AAc) is around 0.05. In yet a preferred embodiment, the mass ratio of hydroxypropyl methylcellulose to acrylic acid (HPMC:AAc) is 1:6 and the mass ratio of crosslinker to acrylic acid (MBA:AAc) is around 0.05. In yet a preferred embodiment, the mass ratio of hydroxypropyl methylcellulose to acrylic acid (HPMC:AAc) is 1:7 and the mass ratio of crosslinker to acrylic acid (MBA:AAc) is around 0.05.

The present invention uses the monomer of AAc as a crosslinker with the polymer to provide “scaffolding support”. The present invention slows down the dissolution of HPMC during the swelling process.

This smart delivery system is fabricated by grafting acrylic acid (AAc) into hydroxypropyl methylcellulose (HPMC) at different ratios to suit different clinical applications. The present invention provides HPMC-AAc hydrogel matrices with different mesh sizes; physicochemical behaviour; and responsiveness to various pH levels that allows for multiple formulations depending on the clinical applications.

The hydrogels of the present invention provide multipurpose, pH-responsive, slow-release delivery systems for the delivery of bioactive compounds. These systems can be used for the systemic and topical delivery of a variety of bioactive compounds, such as therapeutic peptides. The diversity of applications is primarily attributed to the portion of the grafted AAc into the hydrogel system. Manipulation of the level of AAc in the system provides different durability and responsiveness to different pH levels and temperatures. Furthermore, AAc level governs the mesh size or porosity of the network. Subsequently, the diffusion of the bioactive agent will be selected based on the size of the bioactive agent at the equilibrium phase of the swelled polymer.

This type of functionality of the hydrogel leveraging the releasing time of different cargoes, packed in the same system, over a prolonged period. In wound healing, for example, a combination of different bioactive agents such as anti-inflammatories, antibacterial, and epithelialization enhancers can be arrayed in the same dressing ready to be released successively as per their physiological function during the chronological development of the wound during the first week of the injury.

Preferably, the hydrogel matrices of the present invention are highly amorphous. For pharmaceutical applications, this is favourable as amorphous properties are associated with higher internal energy for better reactivity and solubility compared to the crystalline state. Preferably, the hydrogel matrix is amorphous, and more preferably has greater than 85% amorphous character.

The HPMC-AAc hydrogels of the present invention, at a high level of solids, show distinctive glass transitional behaviour with changing temperature altering their mechanical and thermodynamic properties. This state transition is pinpointed by the concept of the glass transition temperature (T_{g}), which can be employed to enhance stability and to prolong shelf life during storage. The T_{g} experimental values were measured using the MDSC technique and small-deformation oscillatory rheology technique. In preferred embodiments of the hydrogel of the invention, when the mass ratio of HPMC:AAc is 1:7, the rheological glass transition temperature (T_{g}) of the hydrogel matrix is around −14° C. In preferred embodiments of the hydrogel of the invention, when the mass ratio of HPMC:AAc is 1:5, the rheological glass transition temperature (T_{g}) of the hydrogel matrix is around 18° C. In preferred embodiments of the hydrogel of the invention, when the mass ratio of HPMC:AAc is 1:3, the rheological glass transition temperature (T_{g}) of the hydrogel matrix is around 40° C.

In preferred embodiments of the hydrogel of the invention, the Lower Critical Solution Temperature (LCST) of the polymerisation reaction is less than 40° C., more preferably less than 39° C., and most preferably is around 38° C.

In preferred embodiments, the hydrogel of the present invention has a solid content of greater than 85%, preferably greater than 86%, still more preferably greater than 87%, still more preferably greater than 88%, and most preferably of around 89%.

A Hydrogel Delivery System for Delivery of a Bioactive Agent

In a further embodiment, the present invention relates to a hydrogel delivery system for delivery of a bioactive agent comprising a hydrogel matrix that is the product of a reaction between hydroxypropyl methylcellulose (HPMC), acrylic acid (AAc) and N,N′-methylenebisacrylamide (MBA).

In preferred embodiments of the delivery system of the invention, the mass ratio of hydroxypropyl methylcellulose to acrylic acid (HPMC:AAc) is between 1:3 and 1:10. More preferably, the mass ratio of hydroxypropyl methylcellulose to acrylic acid (HPMC:AAc) is 1:3 to 1:7. In one preferred embodiment, the mass ratio of hydroxypropyl methylcellulose to acrylic acid (HPMC:AAc) is 1:3. In another preferred embodiment, the mass ratio of hydroxypropyl methylcellulose to acrylic acid (HPMC:AAc) is 1:4. In a further preferred embodiment, the mass ratio of hydroxypropyl methylcellulose to acrylic acid (HPMC:AAc) is 1:5. In yet another preferred embodiment, the mass ratio of hydroxypropyl methylcellulose to acrylic acid (HPMC:AAc) is 1:6. In another preferred embodiment, the mass ratio of hydroxypropyl methylcellulose to acrylic acid (HPMC:AAc) is 1:7.

In further preferred embodiments, the mass ratio of crosslinker to acrylic acid (MBA:AAc) is between 0.01 and 0.10, and more preferably is around 0.05. In one preferred embodiment, the mass ratio of crosslinker to acrylic acid (MBA:AAc) is 0.01. In a further preferred embodiment, the mass ratio of crosslinker to acrylic acid (MBA:AAc) is 0.02. In another preferred embodiment, the mass ratio of crosslinker to acrylic acid (MBA:AAc) is 0.03. In another preferred embodiment, the mass ratio of crosslinker to acrylic acid (MBA:AAc) is 0.04. In a more preferred embodiment, the mass ratio of crosslinker to acrylic acid (MBA:AAc) is 0.05. In another preferred embodiment, the mass ratio of crosslinker to acrylic acid (MBA:AAc) is 0.06. In a further preferred embodiment, the mass ratio of crosslinker to acrylic acid (MBA:AAc) is 0.07. In another preferred embodiment, the mass ratio of crosslinker to acrylic acid (MBA:AAc) is 0.08. In a further preferred embodiment, the mass ratio of crosslinker to acrylic acid (MBA:AAc) is 0.09. In another preferred embodiment, the mass ratio of crosslinker to acrylic acid (MBA:AAc) is 0.10.

Preferably, the hydrogel matrix of the delivery system is amorphous, and more preferably has greater than 85% amorphous character.

In preferred embodiments of the hydrogel delivery system of the invention, the Lower Critical Solution Temperature (LCST) of the polymerisation reaction is less than 40° C., more preferably less than 39° C., and most preferably is around 38° C.

In preferred embodiments, the hydrogel delivery system of the present invention has a solid content of greater than 85%, preferably greater than 86%, still more preferably greater than 87%, still more preferably greater than 88%, and most preferably of around 89%.

In preferred embodiments of the hydrogel delivery system of the invention, when the mass ratio of HPMC:AAc is 1:7, the rheological glass transition temperature (T_{g}) of the hydrogel matrix is around −14° C. In preferred embodiments of the hydrogel delivery system of the invention, when the mass ratio of HPMC:AAc is 1:5, the rheological glass transition temperature (T_{g}) of the hydrogel matrix is around 18° C. In preferred embodiments of the hydrogel delivery system of the invention, when the mass ratio of HPMC:AAc is 1:3, the rheological glass transition temperature (T_{g}) of the hydrogel matrix is around 40° C.

In preferred embodiments of the hydrogel delivery system, the bioactive agent is selected from the group consisting of therapeutic peptides, statins, vitamins and antibiotics. More preferably, the bioactive agent is a therapeutic peptide, and is preferably lactoferrin or a lactoferrin analogue.

Method for Synthesizing an HPMC-AAc Hydrogel Matrix

In a still further embodiment, the present invention relates to a method for synthesizing a hydroxypropyl methyl cellulose-acrylic acid (HPMC-AAc) hydrogel matrix, said method comprising the steps of:
preparing a solution of HPMC in aqueous solvent;
adding AAc to the solution of HPMC to form a reaction mixture;
adding N,N′-methylenebisacrylamide (MBA) to the reaction mixture;
adding a radical initiator to the reaction mixture to initiate polymerisation;
allowing polymerisation to proceed for 4 hours at 38° C. to form a hydrogel matrix solution; and
incubating the hydrogel matrix solution in a drying atmosphere at 38° C. to isolate the hydrogel matrix.

Preferably, the concentration of the solution of HPMC in aqueous solvent is between 1% and 8% (w/w) and is preferably around 2% (w/w). In a preferred embodiment, the concentration of the solution of HPMC in aqueous solvent is around 1% (w/w). In a more preferred embodiment, the concentration of the solution of HPMC in aqueous solvent is around 2% (w/w). In a preferred embodiment, the concentration of the solution of HPMC in aqueous solvent is around 3% (w/w). In a further preferred embodiment, the concentration of the solution of HPMC in aqueous solvent is around 4% (w/w). In another preferred embodiment, the concentration of the solution of HPMC in aqueous solvent is around 5% (w/w). In another preferred embodiment, the concentration of the solution of HPMC in aqueous solvent is around 6% (w/w). In another preferred embodiment, the concentration of the solution of HPMC in aqueous solvent is around 7% (w/w). In another preferred embodiment, the concentration of the solution of HPMC in aqueous solvent is around 8% (w/w). Preferably, the aqueous solvent is deionised water.

In preferred embodiments of the method, the mass ratio of hydroxypropyl methylcellulose to acrylic acid (HPMC:AAc) is between 1:3 and 1:10. More preferably, the mass ratio of hydroxypropyl methylcellulose to acrylic acid (HPMC:AAc) is 1:3 to 1:7. In one preferred embodiment, the mass ratio of hydroxypropyl methylcellulose to acrylic acid (HPMC:AAc) is 1:3. In another preferred embodiment, the mass ratio of hydroxypropyl methylcellulose to acrylic acid (HPMC:AAc) is 1:4. In a further preferred embodiment, the mass ratio of hydroxypropyl methylcellulose to acrylic acid (HPMC:AAc) is 1:5. In yet another preferred embodiment, the mass ratio of hydroxypropyl methylcellulose to acrylic acid (HPMC:AAc) is 1:6. In another preferred embodiment, the mass ratio of hydroxypropyl methylcellulose to acrylic acid (HPMC:AAc) is 1:7.

In preferred embodiments of the method, the mass ratio of crosslinker to acrylic acid (MBA:AAc) is between 0.01 and 0.10, more preferably around 0.05. In one preferred embodiment, the mass ratio of crosslinker to acrylic acid (MBA:AAc) is 0.01. In a further preferred embodiment, the mass ratio of crosslinker to acrylic acid (MBA:AAc) is 0.02. In another preferred embodiment, the mass ratio of crosslinker to acrylic acid (MBA:AAc) is 0.03. In another preferred embodiment, the mass ratio of crosslinker to acrylic acid (MBA:AAc) is 0.04. In a more preferred embodiment, the mass ratio of crosslinker to acrylic acid (MBA:AAc) is 0.05. In another preferred embodiment, the mass ratio of crosslinker to acrylic acid (MBA:AAc) is 0.06. In a further preferred embodiment, the mass ratio of crosslinker to acrylic acid (MBA:AAc) is 0.07. In another preferred embodiment, the mass ratio of crosslinker to acrylic acid (MBA:AAc) is 0.08. In a further preferred embodiment, the mass ratio of crosslinker to acrylic acid (MBA:AAc) is 0.09. In another preferred embodiment, the mass ratio of crosslinker to acrylic acid (MBA:AAc) is 0.10.

A skilled person will recognise that in some hydrogel systems a different suitable crosslinker could be used in place of N,N′-methylenebisacrylamide (MBA), such as ethylene glycol dimethacrylate (EGDMA).

In preferred embodiments of the method, the radical initiator is potassium persulfate (KPS) and N,N,N′,N′-tetramethylethylenediamine (TEMED). Preferably, the mass ratio of KPS:AAc is between 0.04 and 0.1, more preferably around 0.05. Preferably, the mass ratio of TEMED:AAc is between 0.04 and 0.1, more preferably around 0.05.

A skilled person will recognise that the radical reaction may be initiated by any suitable radical initiator, including for example benzoyl peroxide (BPO) as an oxidiser and ammonium persulfate (APS) as a radical source.

In preferred embodiments of the method of the invention, the Lower Critical Solution Temperature (LCST) of the polymerisation reaction is less than 40° C., more preferably less than 39° C., and most preferably is around 38° C.

In preferred embodiments, the method of the present invention further comprises the step of adding a solution of bioactive agent to the polymerisation reaction.

In preferred embodiments of the method of the invention, the bioactive agent is selected from the group consisting of therapeutic peptides, statins, vitamins, antifungals and antibiotics. More preferably, the bioactive agent is a therapeutic peptide, and even more preferably is lactoferrin or a lactoferrin analogue.

The swelling ratio and other swelling behaviours can be used as a useful approximate indicator of drug release behaviour. In preferred embodiments of the hydrogel matrix delivery system of the invention, when the mass ratio of HPMC:AAc is 1:7, the hydrogel has a maximum swellability degree of around 728% at pH 7.4 after saturation for 72 hours. Preferably, when the mass ratio of HPMC:AAc is 1:5, the hydrogel has a maximum swellability degree of around 646% at pH 7.4 after saturation for 72 hours. Preferably, when the mass ratio of HPMC:AAc is 1:3, the hydrogel has a maximum swellability degree of around 486% at pH 7.4 after saturation for 72 hours. In preferred embodiments of the hydrogel matrix delivery system of the invention, when the mass ratio of HPMC:AAc is 1:7, the hydrogel has a maximum swellability degree of around 96% at pH 2.5 after saturation for 72 hours. Preferably, when the mass ratio of HPMC:AAc is 1:5, the hydrogel has a maximum swellability degree of around 94% at pH 2.5 after saturation for 72 hours. Preferably, when the mass ratio of HPMC:AAc is 1:3, the hydrogel has a maximum swellability degree of around 82% at pH 2.5 after saturation for 72 hours.

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually herein.

### Examples

The invention will now be further explained and illustrated by reference to the following non-limiting examples.

Materials

HPMC powder (composition:hydroxypropoxy content ˜9%, viscosity: ˜15 mPa·s for 2% (w/w) polymer in H₂O at 25° C.), acrylic acid (AAc, 99%), N,N′-methylenebisacrylamide (MBA, 99%), N,N,N′,N′-tetramethylethylenediamine (TEMED, 99%) and potassium persulfate (KPS, 99%) were purchased from Sigma-Aldrich, Sydney, Australia. Mueller-Hinton Agar (MHA) and Mueller-Hinton Broth (MHB) were obtained from Thermo Fisher Scientific, Australia. RRM-Lactoferrin (also referred to herein as peptide-x) was synthesised (to 95% purity) by GL Biochem (Shanghai) Ltd, China.

Hydrogel Preparation

HPMC powder was dissolved in deionized water at a concentration of 2% (w/w) at 60° C. 100 mL of this preparation was added to a round bottom flask and cooled to ambient temperature. An appropriate amount of AAc (HPMC:AAc mass ratio of 1:3, 1:4, 1:5, 1:6 and 1:7) was added to the HPMC preparation followed by the correct amount of MBA (MBA:AAc mass ratio of 0.05). After the mixture was effervesced with N₂ for 25 min, KPS (KPS:AAc=0.05) and TEMED (TEMED:AAc=0.05) were added for the initiation of polymerization between HPMC and AAc. The polymerization process was carried out for 4 h at 38° C. under N₂, followed by an incubation period of 48 h at 37° C. in a drying oven (S.E.M. (S.A.) Pty. Ltd. Laboratory Equipment & Supplies, Magill, South Australia). In order to increase the solid content of the samples and maintain it at approximately 89%, an airtight seal Thermo Scientific™ Nalgene™ transparent polycarbonate classic design desiccator partially filled with silica gel (orange self-indicating, 2.5-6.0 mm, 3-8 mesh, Laboratory Reagent), was used.

Physicochemical Characteristics Tests

The prepared hydrogels were characterised by various methods.

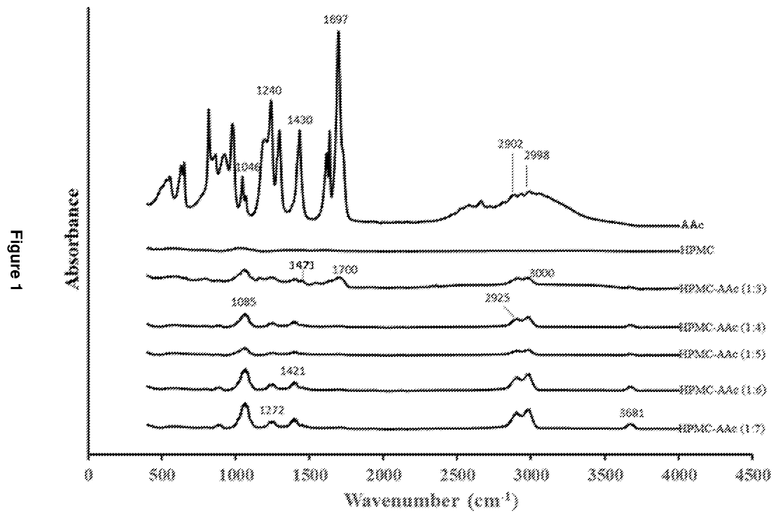

The FTIR spectra of AAc (acrylic acid), HPMC (hydroxypropyl methyl cellulose), and the prepared HPMC:AAc hydrogels (1:7, 1:6, 1:5, 1:4, 1:3, arranged successively downwards) are shown in FIG. 1. The interferograms show new molecular characteristics on the grafted systems. The peak at 3000 cm⁻¹ likely corresponds to N—H stretching vibration (s.v.) shift from 2998 cm⁻¹. The peak at 2925 cm⁻¹ likely corresponds to C—H s.v bond in methyl group with higher intensities than the AAc 2902 cm⁻¹. The peak at 1700 cm⁻¹ for C═O s.v. shifted from 1697 cm⁻¹ and 1471 cm⁻¹ for —C—N shifted from 1430 cm⁻¹. The peak at 1272 cm₋₁ likely corresponds to stretching vibration of C—O—C(ether) bonds. The unaccompanied intense peak at 1085 cm₋₁ likely corresponds to stretched C—O bonds of primary alcohol structure of HPMC.

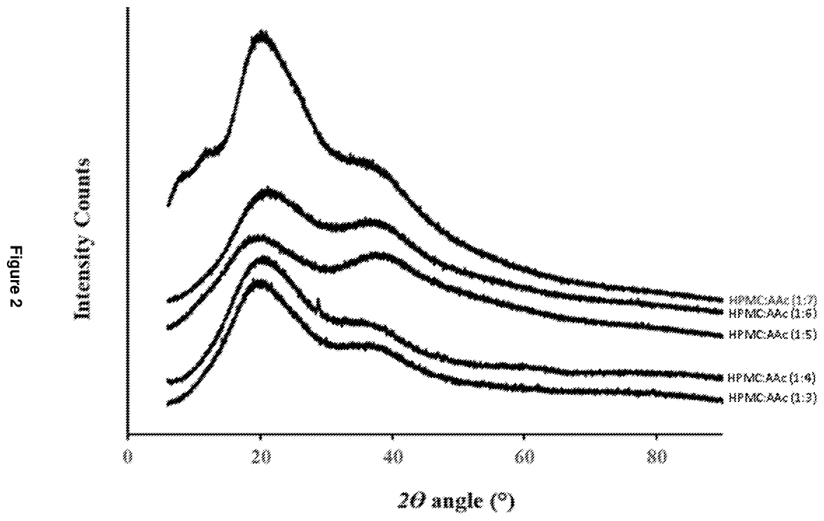

The X-ray diffractogram of the prepared HPMC:AAc hydrogels (1:7, 1:6, 1:5, 1:4, 1:3, arranged successively upwards) are shown in FIG. 2. Smooth and broad peaks were obtained suggesting the prepared hydrogels have an amorphous character. The diffractograms show clearly two molecular events, a peak at 2θ=21° and a shoulder at 37°. The intensity of the event at 2θ=21° was enhanced as the proportion of acrylic acid increased in the binary complex without a significant effect on the observed shoulder at 2θ=37°.

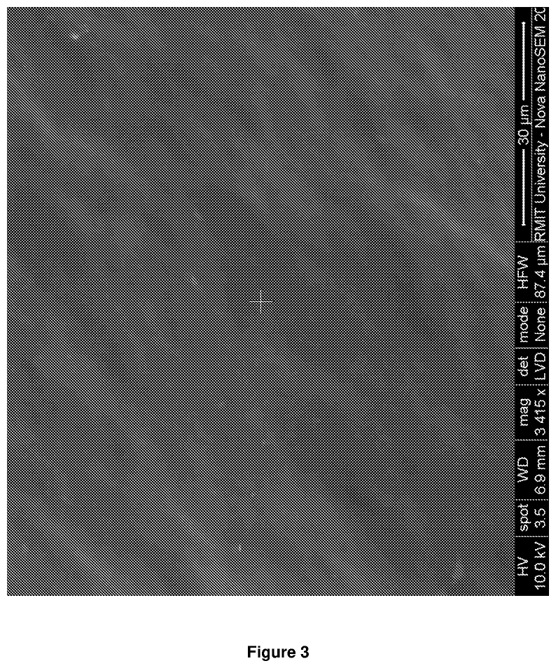

As shown in FIG. 3, a scanning electron micrograph (SEM) images of a prepared HPMC:AAc hydrogel (1:3) shows a smooth surface; a distinctive quality of high level of amorphous structure.

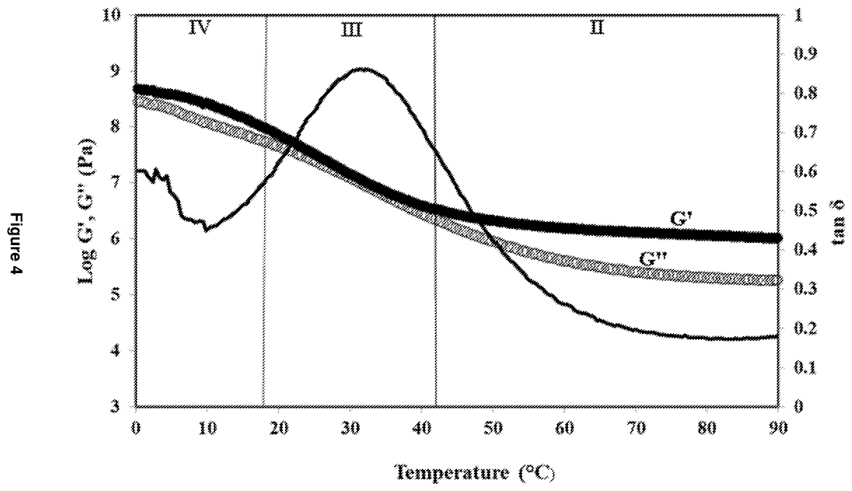

FIG. 4 shows the thermal profiles of G′, G″ and tan δ for one for a prepared HPMC-AAc hydrogel (1:5). Using an AR-G2 rheometer, the correlation between temperature-dependent stress and viscoelasticity was expressed in variation of storage (G′) and loss (G″) modulus. At the high temperature end (region II) the elastic (stored energy) component dominates over the viscous (dissipated energy) component of the network—rubbery plateau of the polymeric network. With the temperature dropping below 42° C., a sharp increase in viscoelasticity (region III). This is the glass transition region—rubber to glass transition over a broad temperature range. At lower experimental temperatures (section IV) the values of storage modulus dominate once more and reach an equilibrium of ca. 109 Pa at 0° C., a glassy state.

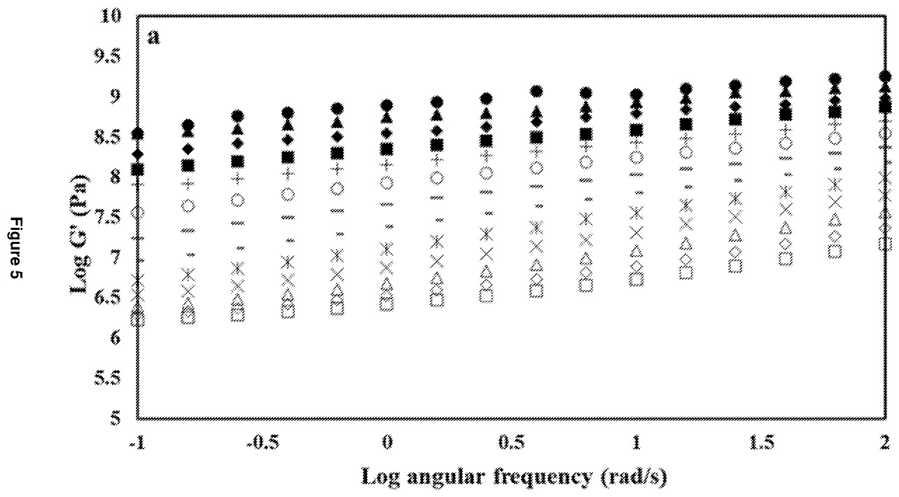

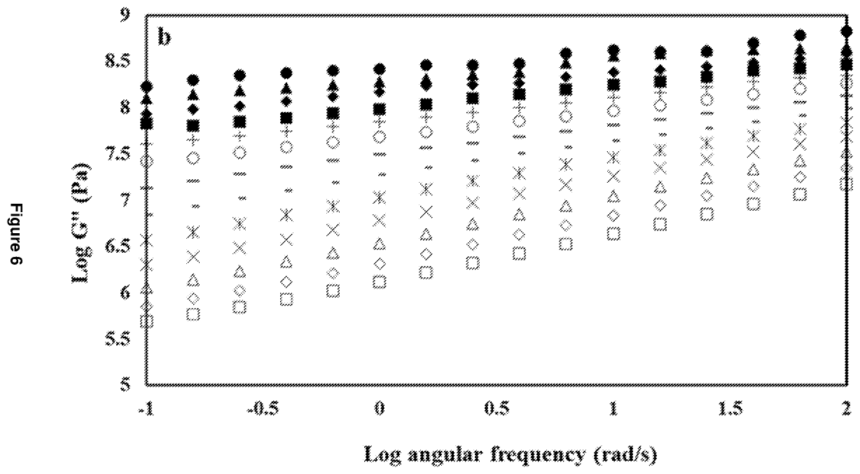

FIG. 5 shows the frequency variation of G′ and FIG. 6 shows the frequency variation of G″. For both plots, the bottom curve is taken at 48° C. (□); other curves successively upward, 44° C. (⋄), 40° C. (Δ), 36° C. (□), 32° C. (□), 28° C. (−), 24° C. (—), 20° C. (∘), 16° C. (+), 12° C. (▪), 8° C. (♦), 4° C. (▴), 0° C. (•), respectively. The mechanical response against the paralleled frequencies at different temperatures (0° C. (•) up to 48° C. (□) demonstrate the effect of time (frequency) and temperature on the mechanical response of the hydrogel. Both G′ & G″ are flat in glass state (e.g. 4° C.) and show high viscosity with increased freq. in glass region (e.g. 32° C.). This is confirmation of the temperature profile of the prepared materials.

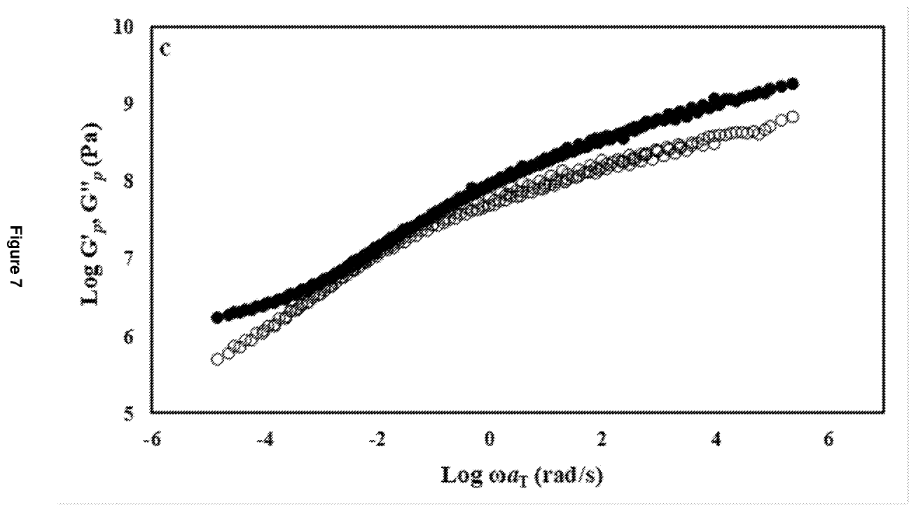

FIG. 7 shows real G′p (•) and imaginary G″p (∘) parts of the complex shear modulus for HPMC-AAc reduced to 20° C. and plotted logarithmically against reduced frequency (ωaT) utilising the mechanical spectra of FIG. 5. This shows the separation between the glassy region and the glassy state with dominating G′ over G″.

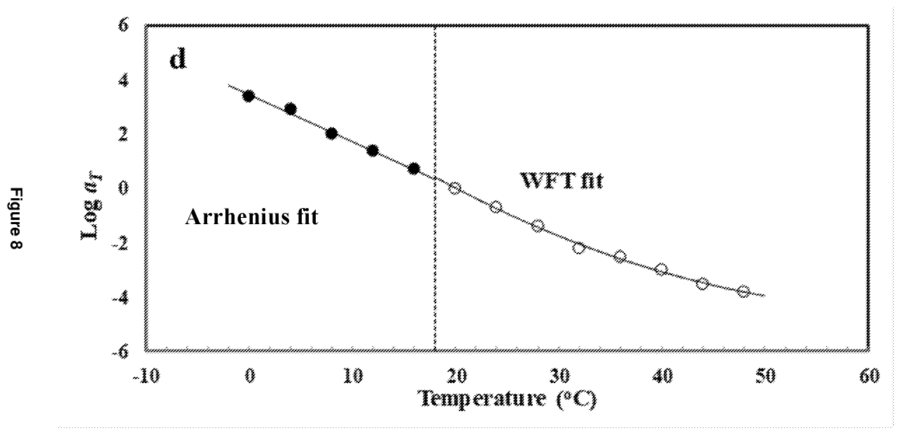

FIG. 8 shows the logarithm of the reduction factor, α_{T}, for HPMC-AAc hydrogel plotted against temperature from the data of the master curve in FIG. 7. The polymer viscoelasticity data fits two different equations. The first equation represents WLF theory (based on the free volume theory) which signifies the glass transition region that separates between the rubbery plateau and the glassy state

$$log {α}_{T} = log [ {G}^{′} ( T ) / {G}^{′} ( T ∘ ) ] = - \frac{( B / 2.303 f ∘ ) ( T - T ∘ )}{( f ∘ / {α}_{f} ) + T - T ∘}$$

The second equation represents the Arrhenius theory (based on the activation energy) which signifies the glassy state (and the rubbery plateau as well)

$${log}_{{α}_{T}} = \frac{{E}_{α}}{2.303 R} ( \frac{1}{T} - \frac{1}{{T}_{0}} )$$

The discontinuity in the temperature variation of shift factors in pinpoints the mechanical glass transition temperature (18° C.) for the HPMC-AAc (1:5) matrix at 89% (w/w) level of solids.

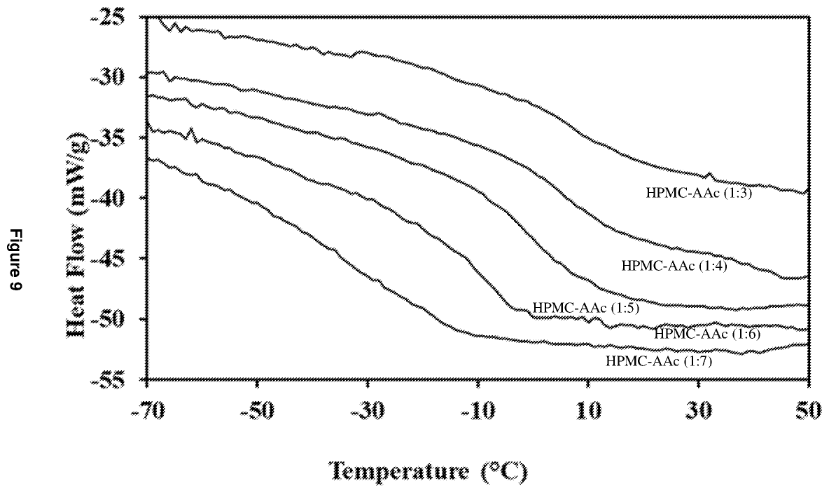

FIG. 9 shows differential scanning calorimetric (DSC) thermogram of the prepared HPMC-AAc hydrogels (1:7, 1:6, 1:5, 1:4, 1:3, arranged successively upwards). The higher the HPMC mass ratio, the lower the thermo-sensitivity of the matrix. For example, the midpoint T_{g} for 1:3 is 13.6° C. compare to −14.2° C. for 1:7 implies more stability due to an enhancement of the hydrophilicity of the matrix (more OH groups). Increasing numbers of polar groups increases intermolecular forces; inter chain attraction and cohesion leading to decrease in free volume resulting in increase in T_{g}.

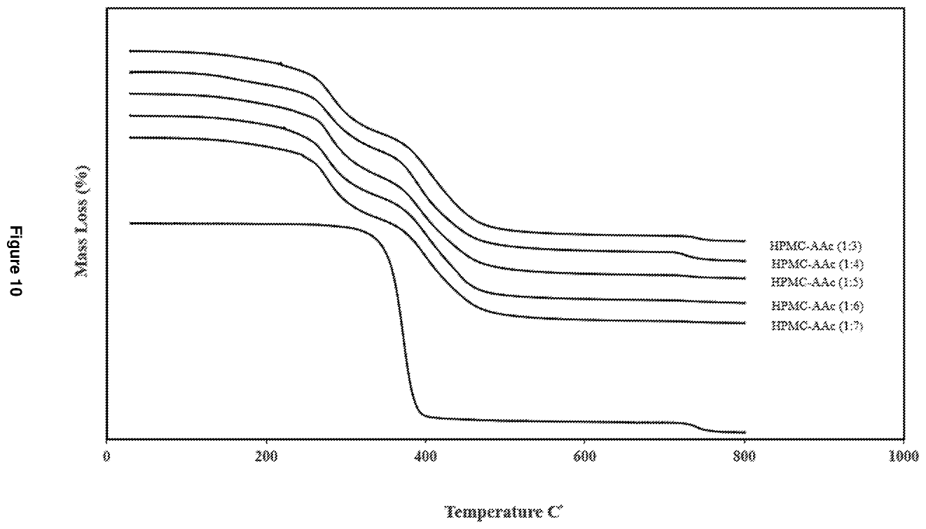

FIG. 10 shows the thermo gravimetric analysis (TGA) of the prepared HPMC-AAc hydrogels (1:7, 1:6, 1:5, 1:4, 1:3, arranged successively upwards), and HPMC powder. In the initial phase of mass alteration was recorded from 40 to 220° C., there was 9% weight loss from bound water known as the intermolecular dehydration reaction. The second stage starts around 220° C. (decarboxylation of AAc) followed by the final stage of HPMC decomposition at 320° C. The weight loss that cascades through 400° C. to reach an equilibrium at temperatures just higher than 500° C. The nitrogen atmosphere was ceased to allow burning of the remaining material at 800° C. (2.3% and 1.0% for HPMC AAc and HPMC, respectively).

Table 1 shows the thermal and rheological glass transition temperatures and Arrhenius and WLF parameters for the prepared HPMC-AAc hydrogels.

TABLE 1
Thermal and rheological glass transition temperatures and Arrhenius and WLF parameters for prepared HPMC-AAc hydrogels

| | DSC T_{g} (° C.) | | | | | | | | |
|---|---|---|---|---|---|---|---|---|---|
| | T_{g} | T_{g} | T_{g} | | Arrhenius | WLF parameters | | | |
| HPMC: | ₒₙₛₑₜ | _{midpoint} | _{endpoint} | Rheological | parameters | | | | α_{f} × 10⁻⁴ |
| AAc | (° C.) | (° C.) | (° C.) | T_{g} (° C.) | Eₐ (kJ) | C₁ | C₂ | f | (deg⁻¹) |
| 1:3 | −2.3 | 13.6 | 22.7 | 40 | 311 | 21.9 | 123.2 | 0.020 | 2 |
| 1:4 | −6.1 | 5.6 | 16.5 | 26 | 206 | 15.6 | 93.8 | 0.030 | 3 |
| 1:5 | −12.9 | −1.1 | 11.5 | 18 | 268 | 21.8 | 119.3 | 0.020 | 2 |
| 1:6 | −18.4 | −8.4 | −4.1 | 12 | 274 | 10.2 | 36.6 | 0.040 | 1.2 |
| 1:7 | −21.5 | −14.2 | −11.7 | −14 | 113 | 12.8 | 39 | 0.030 | 9 |

As Table 1 shows, the higher the content of AAc the lower the network T_{g} and that also supported by the Arrhenius and WLF parameters. The modulated DSC results are also in a broad agreement with the mechanical glass transition temperature.

Swelling Kinetics Tests

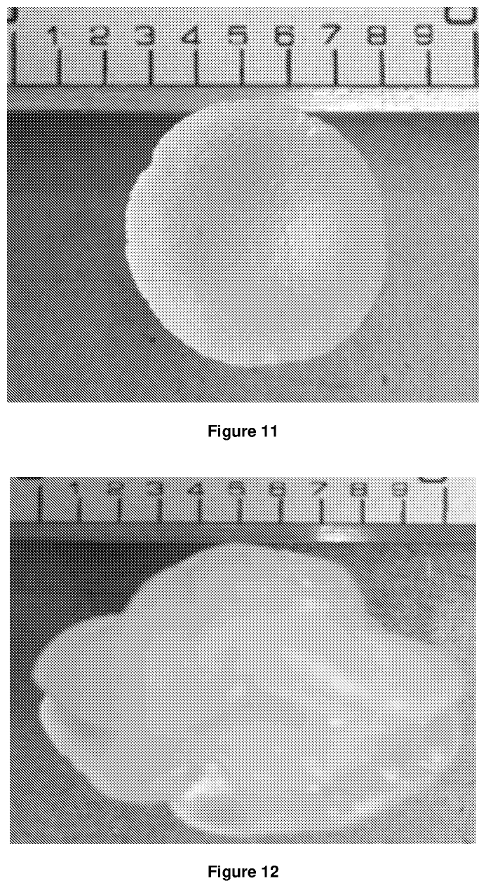

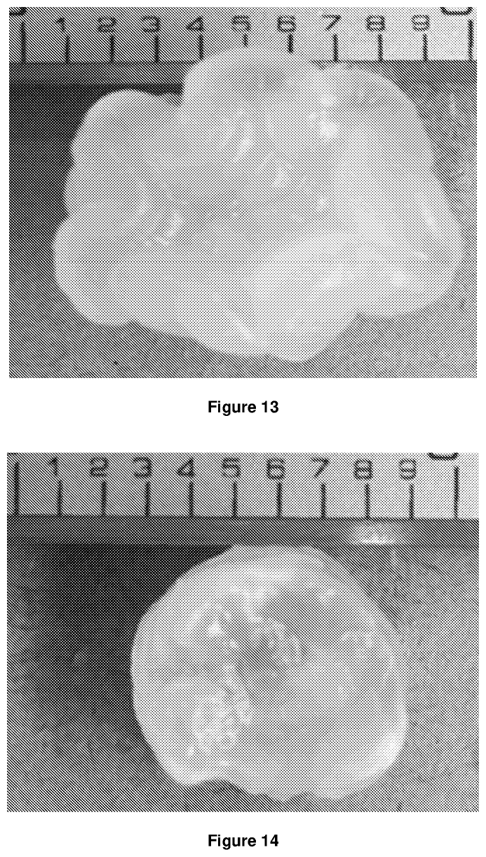

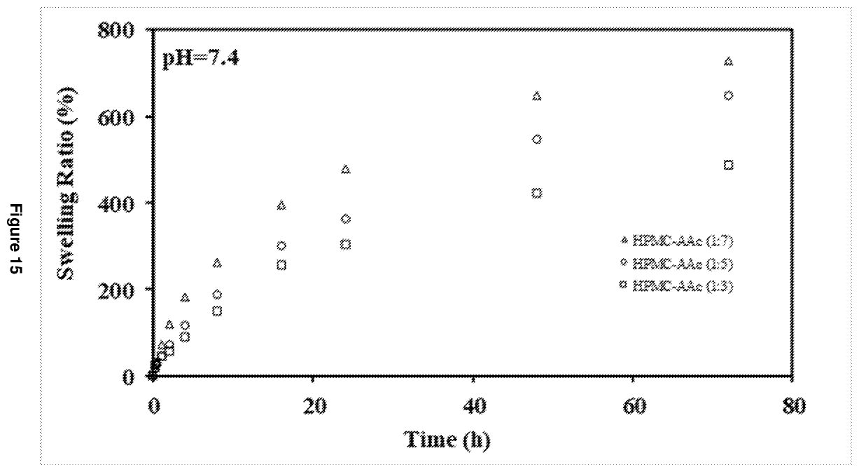

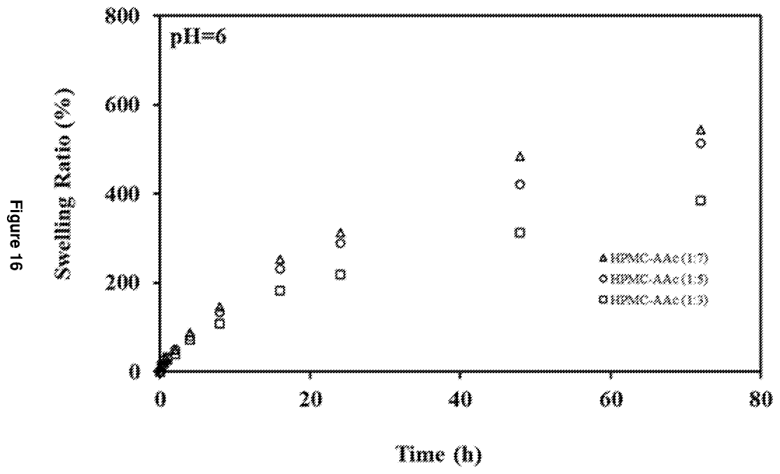

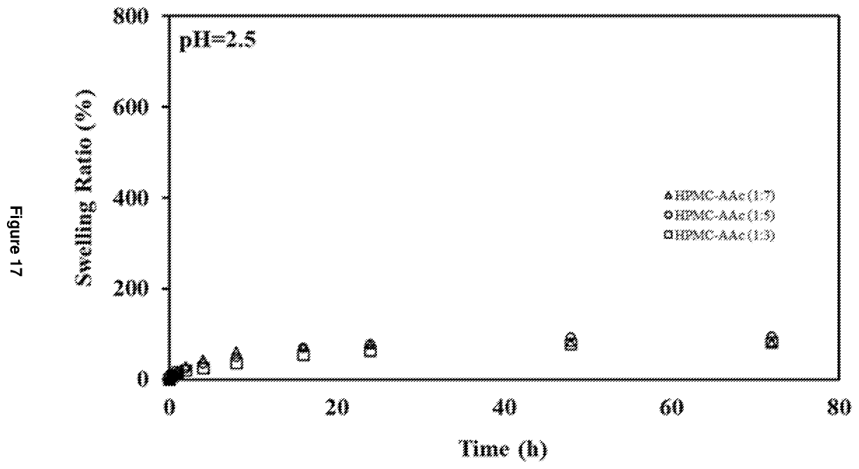

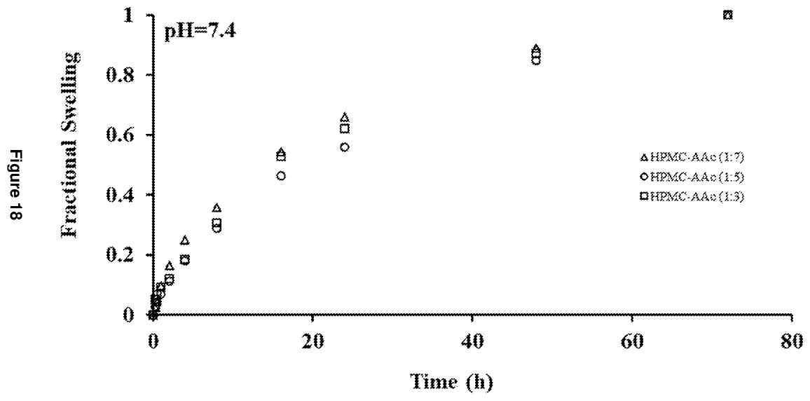

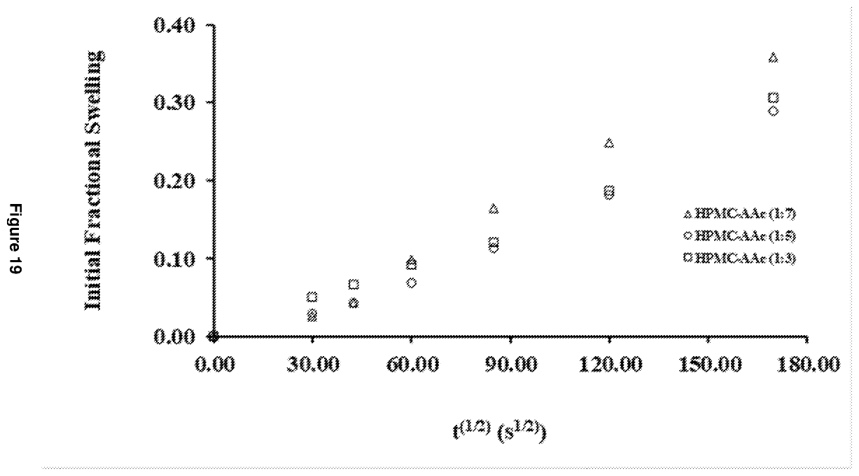

Samples of HPMC:AAc (1:3, 1:5, and 1:7) were prepared in a circular disc form which measures ˜20 mm in diameter, ˜4 mm in thickness, and ˜1.2 g in weight. These samples were incubated in phosphate-buffered saline (PBS) at different pH levels for 24 hours. The measurement of swelling was based on weight changes. The results are shown in FIGS. 11-17 as well as Table 2, below, which shows the swelling exponent (n) and system characteristic constant (k) calculated using power law equation for swelling of HPMC-AAc matrices as a result of water infusion at pH 7.4.

TABLE 2
Swelling exponent (n) and system characteristic constant (k)
calculated using power law equation for swelling of HPMC:AAc
matrices as a result of water infusion at pH 7.4.

| HPMC: | Fractional | Time | | | | |
|---|---|---|---|---|---|---|
| AAc | swelling | range | | | | |
| ratio at | of linear | of linear | | | | Type of |
| pH 7.4 | graph | graph (h) | K | n | R² | swelling |
| 1:7 | 0.00-0.36 | 0-8 | 0.0023 | 0.53 | 0.9834 | Anomalous |
| 1:5 | 0.00-0.29 | 0-8 | 0.0017 | 0.61 | 0.9807 | Anomalous |
| 1:3 | 0.00-0.31 | 0-8 | 0.0017 | 0.67 | 0.9831 | Anomalous |

The swelling tests have shown that the maximum swellability was 728.45% at pH 7.4 for matrix 1:7 compare to 543.60% and 82.55% at pH 6 and pH 2.5 respectively. The matrix with higher level of AAc exhibits the greatest swellability ratio at pH 7.4 (728%) compared with 646.90% and 486.60% for matrix 1:5 and matrix 1:3 respectively.

As shown in FIG. 19, linear relationship is observed between fractional swelling and the square root of exponential time. The data are better linearized via power low equation that yields a variable diffusion exponent: 0.45<n<0.89 or an anomalous kinetics. Relaxation depends on water penetration (both in balance).

Porosity Studies

Samples of HPMC:AAc (1:3, 1:5, and 1:7) were prepared in a circular disc form which measures ˜20 mm in diameter, ˜4 mm in thickness, and ˜1.2 g in weight. The discs were swollen in 50 mL PBS (pH 7.4) until equilibrium swelling was reach at 96 h. Sample weight, diameter and thickness at the start and again at equilibrium were recorded from multiple replicates. Mesh size was calculated using the above parameters, and the molecular weight between crosslinks within HPMC-AAc matrices according to the Flory-Rehner theory.

TABLE 3
Porosity studies for swelling of HPMC-AAc matrices as a result of swelling
with PBS at pH 7.4

| | Volume | Polymer | | | |
|---|---|---|---|---|---|
| | swelling ratio | volume | MW between | Crosslinks | |
| HPMC:AAc | at swelling | fraction of | crosslinks (Mc) | density (q) | Mesh size (ξ) |
| Ratio | state | swelling (v_{2,s}) | (g/mole) | (mole/L) | (nm) |
| 1:7 | 10.59 | 0.104 | 5208 | 13 | 59 |
| 1:5 | 9.39 | 0.119 | 5102 | 18 | 55 |
| 1:3 | 7.05 | 0.165 | 5000 | 21 | 53 |

The molecular weight between adjacent crosslinkers increases as the AAc levels increases and the MBA as a crosslinker (1:7 vs 1:3). This is combined with lower crosslinking density. Consequently, the mesh size increasing as the amount of AAc increases.

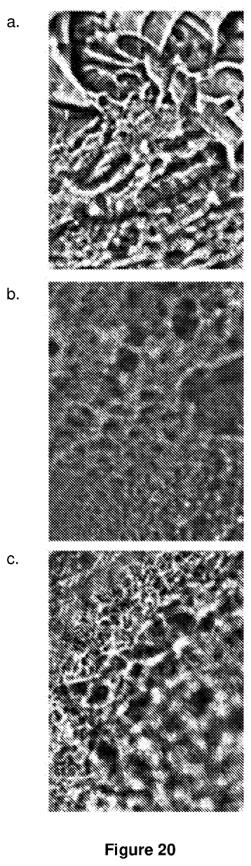

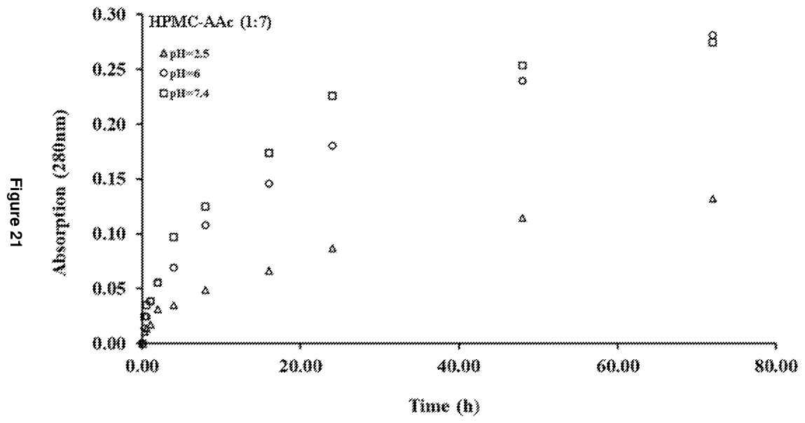

A series of infinity microscopic images of the hydrogels at equilibrium of swelling (96 h) are shown in FIG. 20, particularly (a) HPMC-AAc 1:7, (B) HPMC-AAc 1:5, AND (C) HPMC-AAc 1:3 (10× objective lens).

Diffusion Kinetics Tests

Diffusion Protocol: Samples of HPMC:AAc (1:3, 1:5, and 1:7) were prepared in a cylinder form which measures 13 mm in diameter, 3 mm in thickness, and 9 g in weight. 75 μg of RRM-Lactoferrin peptide (also referred to herein as peptide-x) was added in situ to the hydrogels during polymerization at 37° C. and solidity of 89% has been achieved after an incubation period of 48 h at 37° C. in a drying oven. Diffusions have been tested in an alkaline and acidic PBS solutions. According to the calibration curve the absorption level of 75 μg of the peptide is 3 (280 nm).

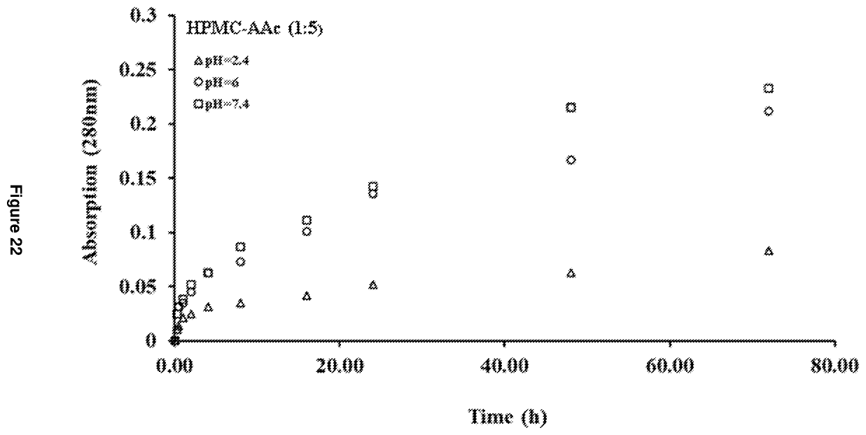

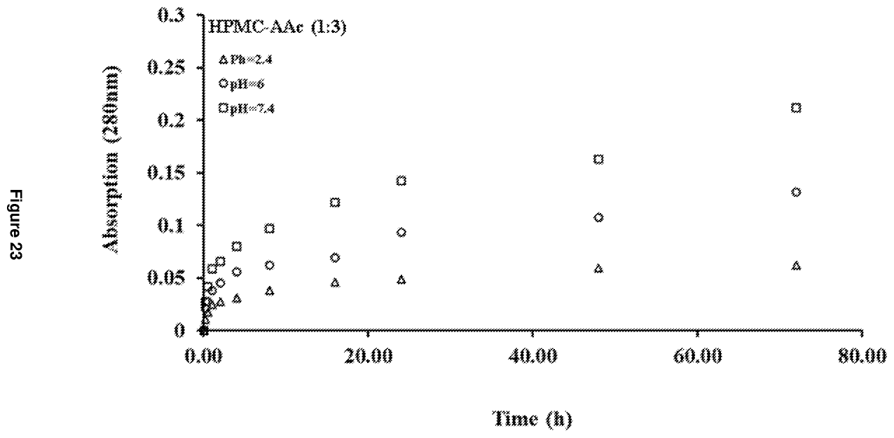

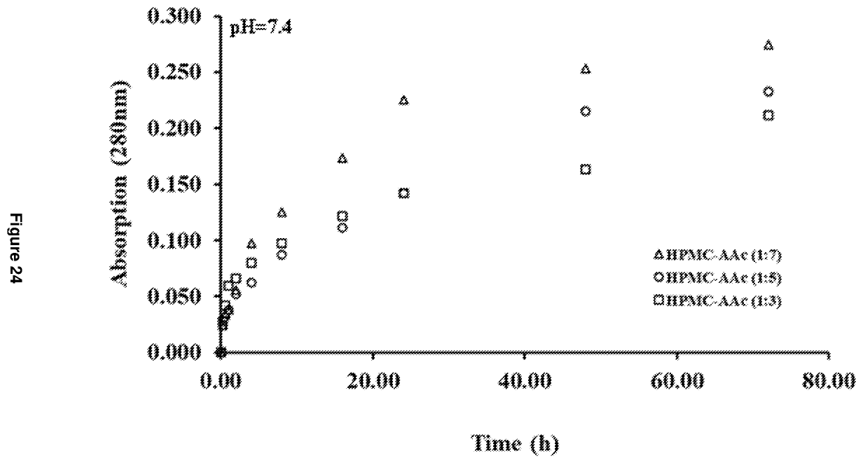

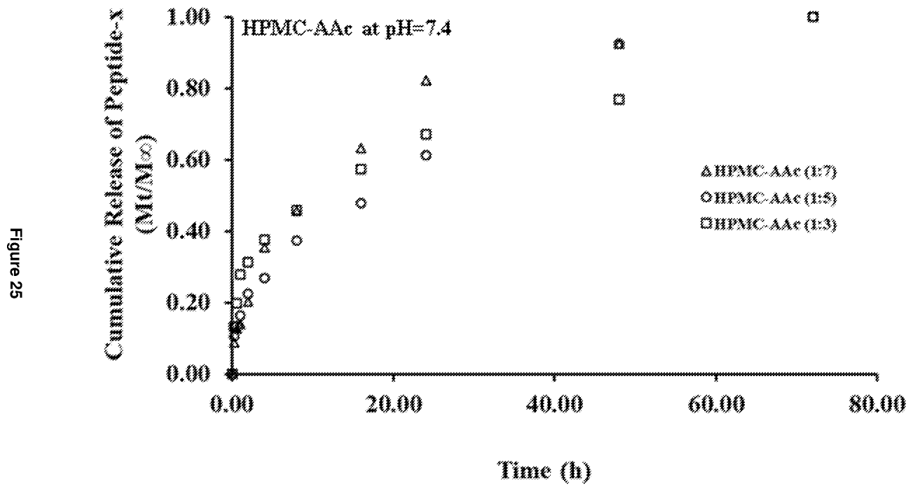

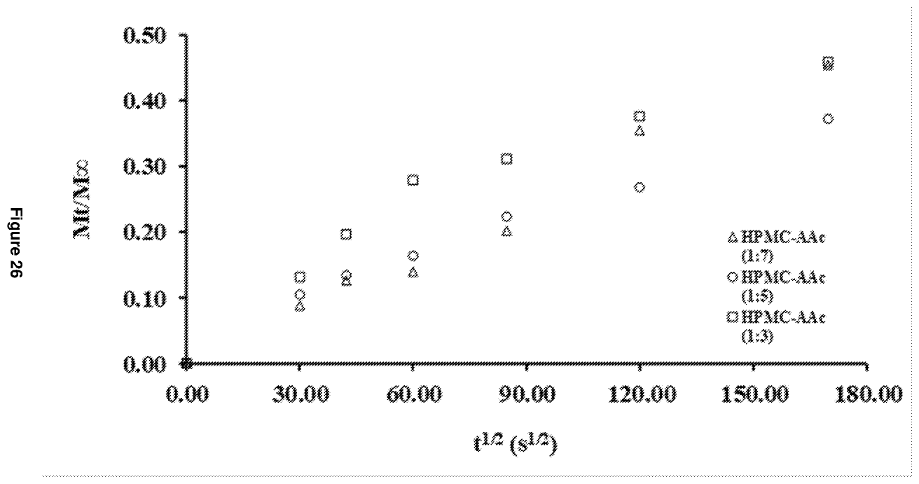

FIGS. 21, 22 and 23 show the peptide diffusion behaviour of different HPMC-AAc 1:7 mass ratios (1:7, 1:5 and 1:3 respectively) at different pH levels. The measurement of peptide release was based on cumulative absorption at 280 nm. The highest accumulative fractional diffusion of the peptide was 2.74 out of 3 at pH 7.4 and released by the 1:7 matrix. FIG. 24 shows the peptide release behaviour by different HPMC-AAc matrices at pH 7.4. FIG. 25 shows the cumulative fractional diffusion of peptide-x analogue by different HPMC-AAc over prolonged period.

TABLE 4
Diffusion exponent (n) and system characteristic constant (k)
calculated using power law equation for diffusion of peptide-x
analogue from HPMC:AA cmatrices at pH 7.4.

| | Fractional | Time | | | | |
|---|---|---|---|---|---|---|
| HPMC: | diffusion | range | | | | |
| AAc | of | of linear | | | | |
| ratio at | linear | graph | | | | Type of |
| pH 7.4 | graph | (h) | K | n | R² | diffusion |
| 1:7 | 0.00-0.46 | 0-8 | 0.0027 | 0.68 | 0.9844 | Anomalous |
| 1:5 | 0.00-0.37 | 0-8 | 0.0021 | 0.71 | 0.9782 | Anomalous |
| 1:3 | 0.00-0.47 | 0-8 | 0.0026 | 0.63 | 0.9256 | Anomalous |

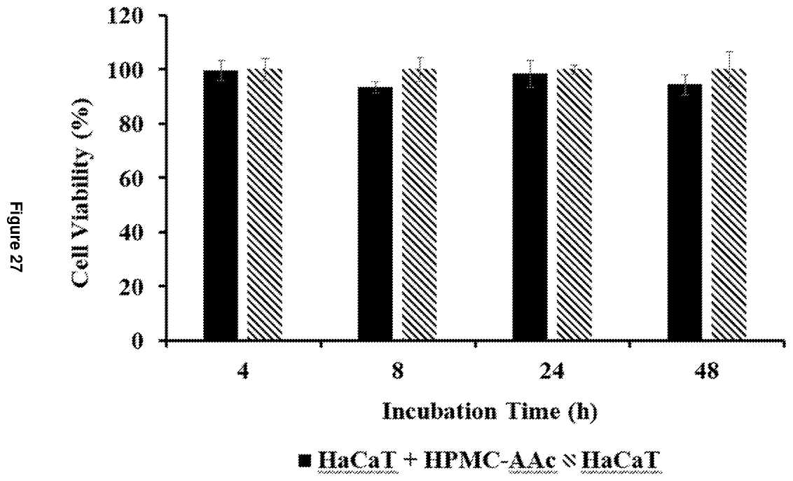

The initial fractional diffusion (i.e. the first 8 hours) was used to determine the type of diffusion by employing the Power Low equation Mt/M∞=Ktⁿ. As FIG. 26 shows, a linear relationship is observed between fractional diffusion and the square root of exponential time. The data are better linearized via power low equation that yields a variable diffusion exponent: 0.45<n<0.89. This anomalous or non-Fickian relationship implies relaxation of the gel governs the diffusion kinetics.

The testing swelling and diffusion kinetics demonstrate that the intestinal pH (˜pH 7.4) is the optimal ambient environment for the relaxation of the biopolymer and the diffusion of the peptide over an elongated period. The higher AAc mass ratios (e.g. HPMC:AAc 1:7) produced greater relaxation and diffusion responses. The diffusion is primarily relaxation-dependent rather than moving boundaries and it follows an anomalous type or zero-order kinetics.

Cytotoxicity Tests

Cytotoxicity tests were conducted on human epithelial colorectal adenocarcinoma (CaCo2) cell line, which mimics the first contact of epithelial cells in the digestive system. Similarly, the cytotoxic effect on human keratinocyte (HaCaT) cell line was investigated, as the first point of contact with the hydrogel should the hydrogel be used in foam-based dressing for wound management. These cell lines were proliferated in 75 cm 2 tissue culture flasks containing DMEM medium. For experiments, cell lines were seeded in 96 well microtiter plates and incubated at 37° C. with 5% CO2 for an overnight before administering the hydrogel. PrestoBlue™ Cell Viability Reagent was utilised over prolonged time to quantitatively measure cell proliferation.

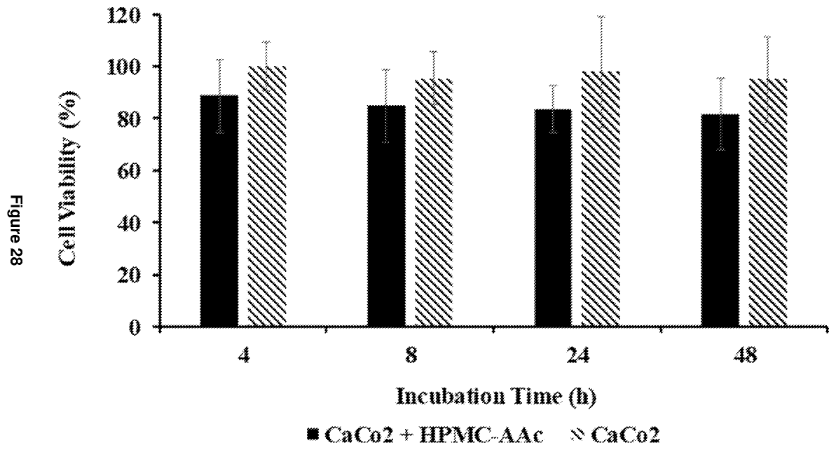

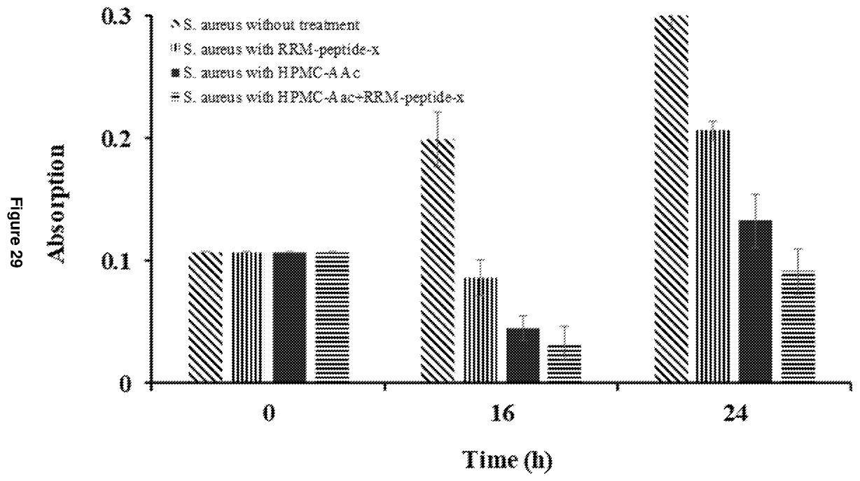

FIG. 27 shows the cell viability of human keratinocyte cells (HaCat) following prolonged incubation with HPMC-AAc (1:7) using PrestoBlue methodology. FIG. 28 shows the cell viability of human epithelial colorectal adenocarcinoma cells (CaCo2) following prolonged incubation with HPMC-AAc (1:7) using PrestoBlue methodology. After 48 hours, the treated cells in both experiments remain viable, demonstrating that the hydrogels are not cytotoxic. This study demonstrates the hydrogels are safe on the HaCaT and the CaCo2 mammalian cell cultures, indicating that the biopolymer is safe to be used clinically

Bioactivity Tests of Peptide-x after Diffusion

The biocompatibility between the prepared hydrogels and the released peptide was investigated. This was evaluated by the bactericidal activity of the released peptide-x from the hydrogel at pH 7.4. Bacterial cultures for two different pathogens were separately tested. The Gram-negative rod bacterium Pseudomonas aeruginosa, and the Gram-positive cocci bacterium Staphylococcus aureus have been exposed to the released peptide over prolonged time.

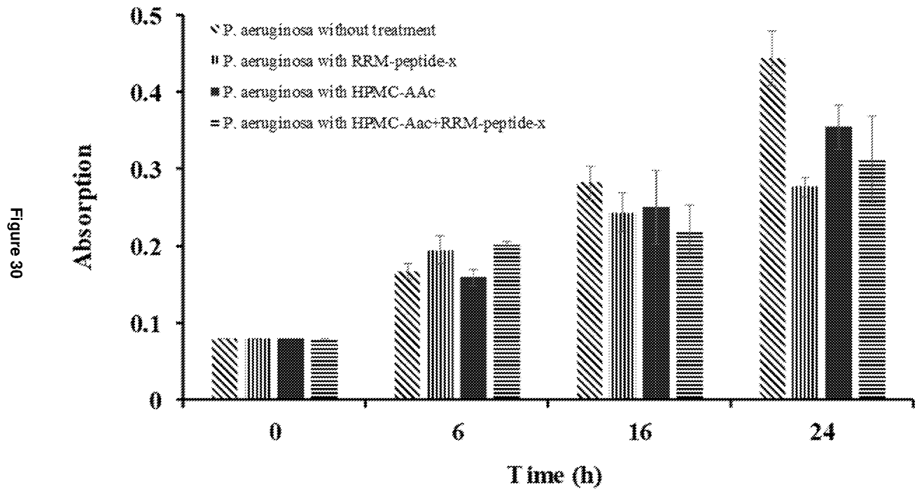

FIG. 29 shows the optical density (O.D.600) readings for Staphylococcus aurous as a response to the released peptide-x from HPMC-AAc biopolymer over an elongated period. As FIG. 29 shows, after both 16 and 24 hours, the sample of S. aureus treated with the HPMS-AAc+RRM-peptide-x has the lowest optical density.

FIG. 30 shows the optical density (O.D.600) readings for Pseudomonas aeruginosa as a response to the released peptide-x from HPMC-AAc biopolymer over an elongated period. As FIG. 30 shows, after 6, 16 and 24 hours, the sample of P. aeruginosa treated with the HPMS-AAc+RRM-peptide-x has an optical density equivalent to the sample treated with RRM-peptide-x.

These results indicate significant antibacterial activity of the released peptide over an extended period in comparison to the untreated samples. These findings indicate that the hydrogel can carry, protect, and timely release the cargo. Moreover, because skin wound infections are primarily caused by the proliferation of Pseudomonas aeruginosa and Staphylococcus aureus in the wound bed, it suggests that the prepared hydrogels can be used in the management and treatment of infected skin wounds.

Those skilled in the art will appreciate that the invention described herein is susceptible to variations and modifications other than those specifically described. It is understood that the invention includes all such variations and modifications which fall within the spirit and scope of the present invention.

1. A hydrogel that is the product of a reaction between hydroxypropyl methyl cellulose (HPMC) and acrylic acid (AAc) crosslinked with N,N′-methylenebisacrylamide (MBA).

2. A hydrogel delivery system for delivery of a bioactive agent comprising a hydrogel matrix that is the product of a reaction between hydroxypropyl methylcellulose (HPMC), acrylic acid (AAc) and N,N′-methylenebisacrylamide (MBA).

3. The hydrogel according to claim 1, wherein the mass ratio of hydroxypropyl methylcellulose to acrylic acid (HPMC:AAc) is between 1:3 and 1:10.

4. The hydrogel according to claim 1, wherein the mass ratio of crosslinker to acrylic acid (MBA:AAc) is between 0.01 and 0.10.

5. The hydrogel according to claim 1, wherein the Lower Critical Solution Temperature (LCST) of the polymerisation reaction is less than 40° C.

6. The hydrogel according to claim 1, wherein the hydrogel matrix is amorphous, and preferably has greater than 85% amorphous character.

7. The hydrogel according to claim 1, wherein when the mass ratio of HPMC:AAc is 1:7, the rheological glass transition temperature (T_{g}) of the hydrogel matrix is around −14° C.

8. The hydrogel according to claim 1, wherein when the mass ratio of HPMC:AAc is 1:5, the rheological glass transition temperature (T_{g}) of the hydrogel matrix is around 18° C.

9. The hydrogel according to claim 1, wherein when the mass ratio of HPMC:AAc is 1:3, the rheological glass transition temperature (T_{g}) of the hydrogel matrix is around 40° C.

10. The hydrogel according to claim 1, wherein the solid content of the hydrogel is greater than 85%.

11. The hydrogel delivery system according to claim 2, wherein the bioactive agent is selected from the group consisting of therapeutic peptides, statins, vitamins and antibiotics.

12. The hydrogel delivery system according to claim 11, wherein the bioactive agent is a therapeutic peptide selected from lactoferrin or a lactoferrin analogue.

13. A method for synthesizing a hydroxypropyl methyl cellulose-acrylic acid (HPMC-AAc) hydrogel matrix, said method comprising the steps of:
preparing a solution of HPMC in aqueous solvent;
adding AAc to the solution of HPMC to form a reaction mixture;
adding N,N′-methylenebisacrylamide (MBA) to the reaction mixture;
adding a radical initiator to the reaction mixture to initiate polymerisation;
allowing polymerisation to proceed for 4 hours at 38° C. to form a hydrogel matrix solution;
incubating the hydrogel matrix solution in a drying atmosphere at 38° C. to isolate the hydrogel matrix.

14. The method according to claim 13, wherein the concentration of the solution of HPMC in aqueous solvent is between 1% and 8% (w/w).

15. The method according to claim 13, wherein the mass ratio of hydroxypropyl methylcellulose to acrylic acid (HPMC:AAc) is between 1:3 and 1:10.

16. The method according to claim 13, wherein the mass ratio of crosslinker to acrylic acid (MBA:AAc) is between 0.01 and 0.10.

17. (canceled)

18. The method according to claim 13, wherein the radical initiator is potassium persulfate (KPS) and N,N,N′,N′-tetramethylethylenediamine (TEMED).

19. The method according to claim 18, wherein the mass ratio of KPS:AAc or the mass ratio of TEMED:AAc is between 0.04 and 0.1.

20. (canceled)

21. The method according to claim 13, further comprising the step of adding a solution of a bioactive agent to the polymerisation reaction, wherein the bioactive agent is selected from the group consisting of therapeutic peptides, statins, vitamins, antifungals and antibiodics.

22. (canceled)

23. The method according to claim 21, wherein the bioactive agent is a therapeutic peptide selected from lactoferrin or a lactoferrin analogue.
